SEQUENCE LISTING (10 sequences)

SEQ ID NO: 1 - DNA, 36 bp, Artificial sequence
  Synthetic probe
aaaaaggattacctataactgtagactcggcttggg

SEQ ID NO: 2 - DNA, 35 bp, Artificial sequence
  Synthetic probe
aaaaacgctcaaatcatcgaggaaaaccgtatgag

SEQ ID NO: 3 - DNA, 60 bp, Artificial sequence
  Synthetic probe
aaaaaagcgctacacacgctcaaatcatcgaggaaaaccgtatgagaaacggatctaagc

SEQ ID NO: 4 - DNA, 30 bp, Artificial sequence
  Synthetic probe
aaaaacgatcaaggagttcttcggcaccag

SEQ ID NO: 5 - DNA, 23 bp, Artificial sequence
  Synthetic probe
ctataactgtagactcggcttgg

SEQ ID NO: 6 - DNA, 22 bp, Artificial sequence
  Synthetic probe
tcatcgaggaaaaccgtatgag

SEQ ID NO: 7 - DNA, 23 bp, Artificial sequence
  Synthetic probe
  misc_feature (9)..(10): /ZEN/ internal quencher
aagagcttttgcggcgtcgtatc

SEQ ID NO: 8 - DNA, 22 bp, Artificial sequence
  Synthetic probe
ggcaggttggtatcaaggttac

SEQ ID NO: 9 - DNA, 22 bp, Artificial sequence
  Synthetic probe
cctaagggtgggaaaatagacc

SEQ ID NO: 10 - DNA, 20 bp, Artificial sequence
  Synthetic probe
actgggcatgtggagacaga

(19) United States
(12) Patent Application Publication
(10) Pub. No.: US 2021/0292746 A1
(43) Pub. Date: Sep. 23, 2021

(54) ENHANCED CAPTURE OF TARGET NUCLEIC ACIDS

(71) Applicant: Northwestern University, Evanston, IL (US)

(72) Inventors: Sally M. McFall, Evanston, IL (US); Jennifer L. Reed, Chicago, IL (US)

(21) Appl. No.: 17/266,430

(22) PCT Filed: Aug. 6, 2019
(86) PCT No.: PCT/US2019/045287

§ 371 Date: Feb. 5, 2021

Related U.S. Application Data
(60) Provisional application No. 62/715,088, filed on Aug. 6, 2018.

(51) Int. Cl. C12N 15/10; C12Q 1/6806

(52) U.S. Cl. CPC C12N 15/1013; C12Q 1/6806

(57) ABSTRACT

Provided herein are methods for enhancing the speed and/or efficiency of a nucleic acid capture using an affinity-tagged capture oligonucleotide (e.g., biotinylated DNA oligo) and an immobilized capture agent (e.g., immobilized avidin/streptavidin). In particular, experiments conducted during development of embodiments herein demonstrate that streptavidin capture of biotinylated nucleic acid complexes occurs more quickly and efficiently at elevated temperatures while simultaneously reducing the amount of nonspecific DNA captured.

### FIELD

Provided herein are methods for enhancing the speed and/or efficiency of a nucleic acid capture using an affinity-tagged capture oligonucleotide (e.g., biotinylated DNA oligo) and an immobilized capture agent (e.g., immobilized avidin/streptavidin). In particular, experiments conducted during development of embodiments herein demonstrate that streptavidin capture of biotinylated nucleic acid complexes occurs more quickly and efficiently at elevated temperatures while simultaneously reducing the amount of nonspecific DNA captured.

### BACKGROUND

The capture of the small molecule biotin by avidin or streptavidin proteins is a widely used and powerful tool that has been adapted for many uses in biology by making use of the strongest reported non-covalent ligand-receptor interaction reported (Kₐ˜4×10¹⁴M⁻¹) (Green, 1990; herein incorporated by reference in its entirety). Streptavidin paramagnetic particles (PMPs), such as Dynal M270 (ThermoFisher Scientific catalog number 65305), are coated with a monolayer of streptavidin providing, for example, 2-3 biotin binding sites per molecule. The Dynal M270 streptavidin PMP manual (Publication No. MAN0008449; herein incorporated by reference in its entirety) states that biotin-streptavidin binding be performed at room temperature for 10-15 minutes and that the length of time required for binding is dependent upon the length of the nucleic acid being captured.

### SUMMARY

Provided herein are methods for enhancing the speed and/or efficiency of a nucleic acid capture using an affinity-tagged capture oligonucleotide (e.g., biotinylated DNA oligo) and an immobilized capture agent (e.g., immobilized avidin/streptavidin). In particular, experiments conducted during development of embodiments herein demonstrate that streptavidin capture of biotinylated nucleic acid complexes occurs more quickly and efficiently at elevated temperatures while simultaneously reducing the amount of nonspecific DNA captured.

In some embodiments, provided herein are methods of capturing a target nucleic acid, comprising: (a) contacting a sample comprising the target nucleic acid and with an affinity-tagged capture oligonucleotide, wherein the capture oligonucleotide is complementary to all or a portion of the target nucleic acid; (b) incubating the sample under conditions that allow a capture/target complex to form between the capture oligonucleotide and the target nucleic acid; (c) contacting the sample and affinity-tagged capture oligonucleotide with a capture agent, wherein the capture agent and the affinity-tag are capable of forming a stable non-covalent complex; (d) incubating the sample at a temperature of 30-85° C.; and (e) separating the capture agent from the sample, thereby removing capture/target complex from the sample. In some embodiments, the affinity tag comprises biotin. In some embodiments, the capture agent comprises streptavidin. In some embodiments, step (b) is performed at a temperature of less than 75° C. In some embodiments, the capture agent is immobilized on a solid support. In some embodiments, the capture agent is covalently linked to the solid support. In some embodiments, the solid support is selected from the group consisting of a well, a tube, a plate, a chip, a bead, a particle, a membrane and a matrix. In some embodiments, the solid support is a paramagnetic particle (PMP). In some embodiments, the capture oligonucleotide is partially complementary to all or a portion of the target nucleic acid. In some embodiments, the incubating of step (d) is carried out for at least 30 seconds. In some embodiments, the incubating of step (d) is carried out for 30 seconds to 48 hours (e.g., 30 seconds, 45 seconds, 1 minute, 2 minutes, 3 minutes, 4 minutes, 5 minutes, 6 minutes, 7 minutes, 8 minutes, 9 minutes, 10 minutes, 30 minutes, 1 hour, 2 hours, 4 hours, 6 hours, 12 hours 24 hours, 48 hours, or ranges therebetween). In some embodiments, methods further comprise washing the capture/target complex following step (e). In some embodiments, methods further comprise eluting the target nucleic acid from the capture/target complex following step (e).

In some embodiments, provided herein are methods of capturing a target nucleic acid, comprising: (a) contacting a sample comprising the target nucleic acid and with a biotin-tagged capture oligonucleotide, wherein the capture oligonucleotide is complementary to all or a portion of the target nucleic acid; (b) incubating the sample under conditions that allow hybridization of the capture oligonucleotide to the target nucleic acid; (c) contacting the sample and biotin-tagged capture oligonucleotide with streptavidin immobilized on a solid surface; (d) incubating the sample at a temperature of 30-85° C. for sufficient time to allow formation of a biotin/streptavidin complex; (e) separating the solid surface from the sample; and (f) washing the solid surface to remove residual sample and/or contaminants. In some embodiments, methods further comprise (g) eluting the target nucleic acid. In some embodiments, methods further comprise (h) amplifying the target nucleic acid after elution from the solid surface (e.g., disturbing the hybridization of the capture oligonucleotide and the target nucleic acid). In some embodiments, methods further comprise (g) amplifying the target nucleic acid without elution from the solid surface.

In some embodiments, step (b) is performed at a temperature of less than 75° C. In some embodiments, the streptavidin is covalently linked to the solid surface. In some embodiments, the solid surface is selected from the group consisting of a well, a tube, a plate, a chip, a bead, a particle, a membrane and a matrix. In some embodiments, the solid surface is a paramagnetic particle (PMP). In some embodiments, the capture oligonucleotide is partially complementary to all or a portion of the target nucleic acid. In some embodiments, the incubating of step (d) is carried out for at least 30 seconds. In some embodiments, the incubating of step (d) is carried out for 30 seconds to 48 hours (e.g., 30 seconds, 45 seconds, 1 minute, 2 minutes, 3 minutes, 4 minutes, 5 minutes, 6 minutes, 7 minutes, 8 minutes, 9 minutes, 10 minutes, 30 minutes, 1 hour, 2 hours, 4 hours, 6 hours, 12 hours 24 hours, 48 hours, or ranges therebetween).

### BRIEF DESCRIPTION OF THE DRAWINGS

FIG. 1. Percent target captured versus time for reactions performed at 23° C. and 60° C.

FIG. 2. Temperature of binding step vs. percent capture.

FIG. 3. Percent target capture vs. temperature, demonstrating that the reduction in binding at 70° C. is due to loss of hybridization, not reduction of biotin-streptavidin binding.

FIG. 4. Percent target capture vs. temperature.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an oligonucleotide” is a reference to one or more oligonucleotides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum, and the like. Sample may also refer to cell lysates or purified forms of the enzymes, peptides, and/or polypeptides described herein. Cell lysates may include cells that have been lysed with a lysing agent or lysates such as rabbit reticulocyte or wheat germ lysates. Sample may also include cell-free expression systems. Environmental samples include environmental material such as surface matter, soil, water, crystals, and industrial samples. Such examples are not however to be construed as limiting the sample types applicable to the present invention.

As used herein, the term “binding pair” means any two molecules that are known to selectively bind to one another. Such binding may include covalent (“covalent binding pair”) and/or non-covalent (“non-covalent binding pair”) interactions. Examples include, but are not limited to, biotin and avidin; biotin and streptavidin; His₆-tag and Ni; an antibody and its epitope; and the like.

As used herein, the term “affinity tag” refers to a molecular entity that is one member of a binding pair, and selectively forms a stable noncovalent interaction with a corresponding “capture agent” The tag is typically sufficiently small and non-specifically inert to allow attachment of the tag to an oligonucleotide or other molecule without interfering with the structure or function of the oligonucleotide or other molecule.

As used herein, the term “capture agent” refers to a molecular entity that is one member of a binding pair, and selectively forms a stable non-covalent interaction with a corresponding “affinity tag.” The capture agent is typically immobilizable to a solid support without significantly affecting binding affinity for the affinity tag.

As used herein, the term “solid support” is used in reference to any solid or stationary material or object to which reagents such as capture agents, mutant proteins, drug-like molecules, and other test components are or may be attached. Examples of solid supports include microscope slides, wells of microtiter plates, coverslips, beads, particles (e.g., paramagnetic particles), resin, cell culture flasks, as well as many other suitable items. Solid supports may be magnetic, paramagnetic, or non-magnetic.

As used herein, the term “complementary” refers to the topological compatibility or interactive structure of interacting surfaces of a nucleic acid binding pair. Preferred complementary structures have binding affinity for each other, and the greater the degree of complementarity the nucleic acids have for each other, the greater the hybridization between the structures. Two nucleic acids that are complementary are capable of forming a complex under hybridization conditions, but are not necessarily 100% complementary (e.g., forming all Watson-Crick base pairs).

As used herein, the terms “hybridization,” “hybridize,” and linguistic variations thereof refer to the process in which two single-stranded polynucleotides bind non-covalently to form a stable complex (e.g., double-stranded polynucleotide). The resulting (usually) double-stranded polynucleotide is a “hybrid” or “duplex.” “Hybridization conditions” will typically include salt concentrations of approximately less than 1M, often less than about 500 mM and may be less than about 200 mM. A “hybridization buffer” is a buffered salt solution such as 5% SSPE, or other such buffers known in the art. Hybridization temperatures can be as low as 5° C., but are typically greater than 22° C., and more typically greater than about 30-40° C. Hybridizations may be performed under stringent conditions, e.g., conditions under which an oligonucleotide will hybridize to its target sequence but will not hybridize to the other, non-complementary or less-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Exemplary stringent conditions include a salt concentration of at least 0.01M to no more than 1M sodium ion concentration (or other salt), a pH of about 7.0 to about 8.3, and a temperature of 25° C., though a suitable conditions depends on the length and/or GC content of the region hybridized.

As used herein, the terms “selectively binds,” “selective binding,” and the like, refers to a binding reaction of two or more binding partners with high affinity and/or complementarity to ensure selective hybridization under designated assay conditions.

Typically, signal that is due to specific binding may be at least three times the standard deviation of the background signal.

As used herein, the term “complex sample” refers to a sample comprising a large number and variety of different compounds, polymers, macromolecules, complexes, etc. A complex sample may comprise buffers, salts, peptides, polypeptides, proteins (including also enzymes), carbohydrates (complex and simple carbohydrates), lipids, fatty acids, fat, nucleic acids, organelles and other cellular components, etc. Examples of complex samples include cells (e.g., live intact cells), cell lysates, body fluids (e.g., blood (or blood products), saliva, urine, etc.), tissues (e.g., biopsy tissue), cells in 3D culture, cells in tissues, reaction mixtures, etc. In particular embodiments, a complex samples contain a target nucleic as well as additional non-target nucleic acids and/or other contaminants.

As used herein, the term “antibody” refers to a whole antibody molecule or a fragment thereof (e.g., fragments such as Fab, Fab′, and F(ab′)₂, variable light chain, variable heavy chain, Fv, it may be a polyclonal or monoclonal or recombinant antibody, a chimeric antibody, a humanized antibody, a human antibody, etc. As used herein, when an antibody or other entity “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹¹ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “antibody fragment” refers to a portion of a full-length antibody, including at least a portion of the antigen binding region or a variable region. Antibody fragments include, but are not limited to, Fab, Fab′, F(ab′)₂, Fv, scFv, Fd, variable light chain, variable heavy chain, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis. For example, a “Fab” fragment comprises one light chain and the C_{H1} and variable region of one heavy chain. The heavy chain of a Fab molecule cannot form a disulfide bond with another heavy chain molecule. A “Fab” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule. An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen. Other antibody fragments will be understood by skilled artisans.

### DETAILED DESCRIPTION

Provided herein are methods for enhancing the speed and/or efficiency of a nucleic acid capture using an affinity-tagged capture oligonucleotide (e.g., biotinylated DNA oligo) and an immobilized capture agent (e.g., immobilized avidin/streptavidin). In particular, experiments conducted during development of embodiments herein demonstrate that streptavidin capture of biotinylated nucleic acid complexes occurs more quickly and efficiently at elevated temperatures while simultaneously reducing the amount of nonspecific DNA captured.

Sequence-specific capture extraction methods reduce the co-extraction of contaminating DNA (e.g., human DNA), which is a major inhibitor of downstream amplification. In such methods, an affinity-tagged capture oligonucleotide is added to a sample containing a target nucleic acid (e.g., and other non-target nucleic acids). The capture oligonucleotide comprises a sequence that is complementary (or partially complementary) to a sequence within the target nucleic acid. The sample is then combined with a capture agent (e.g., immobilized to a solid surface) capable of forming a stable complex with the affinity tag. Portions of the sample not bound to the capture agent (e.g., including unbound nucleic acids) are washed away, and thereby the target nucleic acid is purified/isolated from the sample. In particular embodiments herein, the affinity tag is biotin and the capture agent is streptavidin. In some embodiments, the capture agent (e.g., streptavidin) is immobilized on a particle (e.g., paramagnetic particle (PMP)) or other solid support.

In some embodiments, biotinylated capture oligonucleotides that have a sequence complementary to a target nucleic acid (e.g., DNA, RNA, etc.) are added to a sample (e.g., comprising lysed cells). The capture oligonucleotide forms a capture/target complex with the target nucleic acid. PMPs coated with streptavidin (e.g., DYNABEADS M-270 Streptavidin (Catalog nos. 65305, 65306)) are added, and the high affinity of biotin and streptavidin allows for the biotinylated capture/target complex to be isolated.

Biotin-streptavidin binding conditions are described in Publication No. MAN0008449 TbermoFisher Scientific; herein incorporated by reference in its entirety. This protocol recommends immobilizing biotinylated nucleic acid complexes onto M270 paramagnetic particles (PMPs) by incubating at room temperature for 15-30 minutes. Other protocols for streptavidin capture of biotin tagged targets (e.g., nucleic acid complexes, non-nucleic acid targets, etc.) describe similar capture conditions, particularly with respect to temperature. Because increased temperature tends to have a detrimental impact on non-covalent interactions, increased temperatures are avoided during binding/capture steps. Experiments conducted during development of embodiments herein demonstrate that incubating a streptavidin-coated solid surface (e.g., PMPs) at elevated temperatures (e.g., 30° C., 35° C., 40° C., 45° C., 50° C., 55° C., 60° C., 65° C., 70° C., 75° C., 80° C., 85° C., or ranges therebetween) improved the yield of biotinylated oligo-genomic DNA complexes, allowed for a reduction in time of the binding step, improved the yield of the target nucleic acid, and yielded less contaminant nucleic acid (e.g., human DNA) via non-specific interactions.

In some embodiments, provided herein are methods of capturing a target nucleic acid from a sample. In some embodiments, the sample is a complex sample. In some embodiments, the sample is a biological sample or an environmental sample. In some embodiments, the sample is a cell lysate. In some embodiments, the sample comprises target nucleic acid. In some embodiments, the sample is suspected of containing target nucleic acid. In some embodiments, the sample comprises non-target nucleic acid (e.g., in addition to target nucleic acid or being suspected of containing target nucleic acid).

In some embodiments, methods provide combining a sample (or a portion of a sample) with a capture oligonucleotide. In some embodiments, a capture oligonucleotide comprises (i) a nucleic acid and (ii) an affinity tag.

In some embodiments, a capture oligonucleotide (e.g., biotin oligonucleotide) that has been hybridized to a target nucleic acid is contacted with a capture agent (e.g., streptavidin bound to a solid surface), and the sample is incubated at a temperature of 30-85° C. (e.g., 30° C., 35° C., 40° C., 45° C., 50° C., 55° C., 60° C., 65° C., 70° C., 75° C., 80° C., 85° C., or ranges therebetween) for a time of 10 seconds to 48 hours (e.g., 10 s, 20 s, 30 s, 45 s, 1 m, 2 m, 4 m, 10 m, 20 m, 30 m, 45 m, 1 h, 2 h, 4 h, 6 h, 12 h, 24 h, 48 h, or ranges therebetween). In particular embodiments, because of the enhanced binding of the affinity tag and capture agent at high temperature, the time of incubation is reduced to between 5 seconds and 10 minutes (e.g., 5 s, 10 s, 15 s, 20 s, 30 s, 45 s, 1 m, 2 m, 4 m, 10 m, or ranges therebetween).

In some embodiments, the nucleic acid portion of the capture oligonucleotide is single stranded. In some embodiments, the nucleic acid is a DNA oligonucleotide or an RNA oligonucleotide. In some embodiments, a capture oligonucleotide comprises a nucleic acid of 6-50 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or ranges therebetween) nucleotides in length. In some embodiments, a capture oligonucleotide is complementary to all or a portion of a target nucleic acid. In some embodiments, at least 50% (e.g., >50%, >55%, >60%, >65%, >70%, >75%, >80%, >85%, >90%, >95%, 100%) of the nucleobases of the capture oligonucleotide are complementary (e.g., capable of forming base pairs, capable of forming Watson-Crick base pairs (e.g., A-T, A-U, G-C), etc.)) to all or a portion of a target nucleic acid.

In some embodiments, the affinity tag of a capture oligonucleotide has a non-covalent binding partner (e.g., a capture agent) that selectively binds to the affinity tag and forms a stable capture complex. In some embodiments, the capture agent and the affinity tag for a stable complex under hybridization conditions, under stringent conditions, etc. In some embodiments, the capture agent and affinity tag pair are selected from biotin and streptavidin; biotin and avidin; hexa-histidine and Ni; an antibody and its epitope; etc. In particular embodiments, the capture agent is streptavidin and the affinity tag is biotin.

In some embodiments, the affinity tag is attached to an appropriate nucleic acid to form a capture oligonucleotide. In some embodiments, the affinity tag and nucleic acid are covalently linked. In some embodiments, an affinity tag is attached to the 3′-end, 5′-end, or internally within an oligonucleotide. In some embodiments, the affinity tag and nucleic acid are linked directly (e.g., via covalent bond) or through an appropriate linker (e.g., PEG linker, alkyl chain, heteroalkyl chain, etc.). In some embodiments, “click” chemistry techniques or other known methods for attachment of tags to molecular entities (e.g., oligonucleotides) find use in the attachment of the affinity tag to form the capture oligonucleotide. In the case of a biotin affinity tag, any suitable method of oligonucleotide biotinylation may be used, for example, via the phosphoramidite method using commercial biotin phosphoramidite, although other known methods of biotinylation find use within the scope herein.

In some embodiments, the capture oligonucleotide is combined with the sample and the combined sample is exposed to conditions (e.g., incubated) to facilitate specific hybridization of the capture oligonucleotide to the target portion of the target nucleic acid. In some embodiments, hybridization conditions are determined based on, for example, the length of the capture oligonucleotide, the length of the target nucleic acid, the ratio of length of the capture oligonucleotide to the length of the target nucleic acid, the GC content of the capture oligonucleotide, the percent complementarity of the capture oligonucleotide and the length of the target nucleic acid. In some embodiments, hybridization conditions are selected to favor specific hybridization of the capture oligonucleotide to the target nucleic acid, while disfavoring non-specific hybridization of the capture oligonucleotide to non-target nucleic acid. In some embodiments, hybridization is carried out at 30-75° C. (e.g., 30° C., 35° C., 40° C., 45° C., 50° C., 55° C., 60° C., 65° C., 70° C., 75° C., or ranges therebetween), with appropriate levels of salt (e.g., Na ions), at suitable pH (e.g., 6.0-8.0), and with suitable buffers and other additives (e.g., DTT, formamide, SDS, etc.). Determination/optimization of hybridization conditions for the capture oligonucleotide and the target nucleic acid is understood in the field.

In some embodiments, methods provide combining a capture agent with a sample (or a portion of a sample) comprising a capture oligonucleotide bound to a target nucleic acid (e.g., a capture/target complex). In some embodiments, a capture agent is provided that is capable of forming a stable complex with the affinity tag of the capture oligonucleotide. In some embodiments, the capture agent binds to the affinity tag with a dissociation constant (K_{d}) of less than 10⁻⁴ mol/L, 10⁻⁵ mol/L, 10⁻⁶ mol/L, 10⁻⁷ mol/L, 10⁻⁸ mol/L, 10⁻⁹ mol/L, 10⁻¹⁰ mol/L, 10⁻¹¹ mol/L, or 10⁻¹² mol/L. In some embodiments, the capture agent is streptavidin and the affinity tag is biotin. In some embodiments, the capture agent is avidin and the affinity tag is biotin. In some embodiments, the capture agent is an avidin or streptavidin variant or polypeptide derived from avidin or streptavidin. In some embodiments, the capture agent is an antibody (or antibody fragment) and the affinity tag comprises an epitope for the antibody (or antibody fragment). In some embodiments, any binding pair capable of forming a stable complex, with one member capable of conjugation to an oligonucleotide (e.g., His₆, etc.), and a second member capable of immobilization on a solid substrate (e.g., Ni, etc.), may find use in embodiments herein.

In some embodiments, the capture agent is immobilized on a solid surface or substrate. Suitable chemistries for immobilization of such agents (e.g., a protein or polypeptide (e.g., streptavidin, etc.), etc.) to a solid surface or substrate (e.g., comprising metal, plastic, or glass) are well understood in the field. Suitable solid surfaces include, but are not limited to: beads (e.g., magnetic beads), wells, chips, tubes, plates, particles, membranes, matrices, paper, etc. In some embodiments, solid surfaces/matrix is made of any suitable materials, such as: Ahlstrom CytoSep, Cellulose nitrate, Cellulose acetate, Cellulose (e.g., Whatman FTA-DMPK-A, B, and C cards; Whatman ET 3/Chr; Whatman protein saver 903 cards; Whatman Grade 1 filter paper; Whatman FTA Elute; Ahlstrom 226 specimen collection paper; etc.), Noviplex Plasma Prep Cards, Polypropylene membrane, PVDF, nitrocellulose membrane (Millipore Nitrocellular Hi Flow Plus) polytetrafluoroethylene film, mixed cellulose esters, Glass fiber media (e.g., Whatman unifilter plates glass fiber filter membrane, Agilent dried matrix spotting cards, Ahlstrom grade 8950, etc.), plastic (e.g., polyester, polypropylene, polythersulfene, poly (methacrylate), acrylic polymers, polytetrafluoreten, etc.), natural and synthetic polymers (e.g., mixture of polymers, co-block polymers, etc.), sugars (e.g., pullulan, trehalose, maltose, sucrose, cellulose, etc.), polyamides (e.g., natural (e.g., wool, silk, etc.), synthetic (e.g., aramids, nylon, etc.), etc.), metals (e.g., aluminum, cadmium, chromium, cobalt, copper, iron, manganese, nickel, platinum, palladium, rhodium, silver, gold, tin, titanium, tungsten, vanadium, zinc, etc.), alloys (e.g., alloys of aluminium (e.g., Al—Li, alumel, duralumin, magnox, zamak, etc.), alloys of iron (e.g., steel, stainless steel, surgical stainless steel, silicon steel, tool steel, cast iron, Spiegeleisen, etc.), alloys of cobalt (e.g., stellite, talonite, etc.), alloys of nickel (e.g., German silver, chromel, mu-metal, monel metal, nichrome, nicrosil, nisil, nitinol, etc.), alloys of copper (e.g., beryllium copper, billon, brass, bronze, phosphor bronze, constantan, cupronickel, bell metal, Devarda's alloy, gilding metal, nickel silver, nordic gold, prince's metal, tumbaga, etc.), alloys of silver (e.g., sterling silver, etc.), alloys of tin (e.g., Britannium, pewter, solder, etc.), alloys of gold (electrum, white gold, etc.), amalgam, etc.), ELISPot plates, Immunoassay plates, Tissue culture plates, etc.

In some embodiments, a solid surface or substrate is coated. A coated solid surface or substrate may be a Langmuir-Bodgett film, functionalized glass, germanium, silicon, PTFE, polystyrene, gallium arsenide, gold, silver, membrane, nylon, PVP, polymer plastics, or any other material known in the art that is capable of having functional groups such as amino, carboxyl, Diels-Alder reactants, thiol or hydroxyl incorporated on its surface. The capture agent may be attached to the coating or such functional groups. In other embodiments, these groups are covalently attached to crosslinking agents for subsequent binding of the capture agent. Typical crosslinking groups include ethylene glycol oligomer, diamines, and amino acids.

In some embodiments, the sample comprising capture-oligonucleotide-bound target nucleic acid is added to a solid substrate (e.g., matrix, tube, well, slide, etc.). In some embodiments, a solid substrate (e.g., bead, particle, etc.) is added to the sample comprising capture-oligonucleotide-bound target nucleic acid.

In some embodiments, the capture agent is attached to the solid surface or substrate by a cleavable linker. In some embodiments, the linker is an enzyme-cleavable, photo-cleavable, or chemically-cleavable linker.

In some embodiments, upon binding of the capture agent (e.g., immobilized on a solid surface or substrate) to the affinity tag (e.g., of the capture oligonucleotide, bound to the target nucleic acid), steps are taken to separate the capture complex (e.g., immobilized on a solid surface or substrate) from the remainder of the sample, contaminants, non-target nucleic acids, etc. In some embodiments, the solid surface or substrate to which the capture complex is bound is removed from the sample. For example, in some embodiments, the solid surface or substrate comprises magnetic beads (e.g., paramagnetic particles (PMPs) and magnetic force is used to pull the immobilized capture complexes from the sample. In other embodiments, such as when the solid surface or substrate is a tube or well, the sample is removed from the solid surface or substrate, leaving the immobilized capture complex behind. In some embodiments, one or more wash steps are performed to remove additional contaminants and/or residual sample from the solid surface and capture complex. Suitable wash solutions are understood in the field, but may include buffers such as Tris (e.g., 10 mM Tris pH 8.0), phosphate buffer, etc., surfactants such as Tween (e.g., 0.01% Tween 20), and other suitable additives. In some embodiments, following separation of the capture complex from the remaining sample/contaminants, the target nucleic acid is eluted from the immobilized capture complex. In some embodiments, elution is performed in an elution buffer. In some embodiments, suitable elution buffers are understood in the field. In some embodiments, elution is performed at elevated temperature (e.g., >70° C., >75° C., >80° C., >85° C., >90° C., etc.). In some embodiments, elution is performed in a buffer that is suitable for further manipulation and/or analysis (e.g., amplification, sequencing, etc.).

In some embodiments, target nucleic acids purified by the methods described herein are suitable for further manipulation and/or analysis. In some embodiments, methods herein comprise manipulation and/or analysis of nucleic acids. Exemplary manipulation and/or analysis techniques include amplification, sequencing (e.g., Next-Gen sequencing), mass spectrometry, hybridization, probing, etc.

In some embodiments, the methods herein find use with one or more devices and methods for the isolation, manipulation, and/or analysis of nucleic acids. Such devices and methods include, but are not limited to, those described in U.S. Pub. No. 2014/0057271; International App. No. PCT/US18/36348; and U.S. Pub. No. 2018/0016623; each of which is herein incorporated by reference in their entireties. In some embodiments, a capture agent is immobilized to a surface of a device. In some embodiments, a capture agent is immobilized to a solid substrate (e.g., a PMP) within a device.

### EXPERIMENTAL

### Assay Conditions

10,000 copies of Chlamydia trachomatis (CT) genomic DNA (Chlamydia trachomatis Serovar E (ATCC® VR-348BD™) was captured in the presence of human buccal cells (2.4 million cells/sample). 300 ul sample in 1% SDS was treated with 30 U proteinase K (Sigma P2308) for 5 minutes at room temperature and then transferred to a Benchmark heater shaker set to 100° C. for 5 minutes to lyse bacteria and melt double stranded DNA. Biotinylated CT ORF3 capture probes 1 and 2 (Table 1) were then hybridized at room temperature (23° C.) or 60° C. 40 ul Dynal M270 Streptavidin coated paramagnetic particles (PMPs) (ThermoFisher Scientific 65305) washed 2× in 10 mM Tris pH 8.0 with 0.01% Tween 20 buffer were added to samples and mixed as described in individual experiments. The streptavidin coated PMPs captured the biotinylated oligo-target DNA complex. Extracted DNA was eluted from PMPs by heating at 75° C. for 3 minutes into 10 ul, all of which was added to qPCR reaction. A standard curve made of CT genomic DNA was used to quantify each experiment's results.

TABLE 1
Oligonucleotides used for PCR and sequence
specific capture.

| | | SEQ |
|---|---|---|
| | | ID |
| | | NO: |
| ORF3 F | cta taa ctg tag act cgg ctt gg | 5 |
| ORF3 R | tca tcg agg aaa acc gta tga g | 6 |
| ORF3 FAM | /56FAM/ AAG AGC TTT /ZEN/ TGC GGC | 7 |
| probe 1 | GTC GTA TC /3IABkFQ/ | |
| ORF3 Cap 1 | Biotin-AAA AAG GAT TAC CTA TAA CTG | 1 |
| | TAG ACT CGG CTT GGG | |
| ORF3 Cap 2 | Biotin- AAA AAC GCT CAA ATC ATC GAG | 2 |
| | GAA AAC CGT ATG AG | |
| BGA F | GGC AGG TTG GTA TCA AGG TTA C | 8 |
| BGA R | CCT AAG GGT GGG AAA ATA GAC C | 9 |
| BGA Quasar | /Q670/ ACT GGGCAT GTG GAG ACA GA | 10 |
| 670 probe | | |

Binding Occurs Faster and Yield is Better when Incubation is at Elevated Temperature

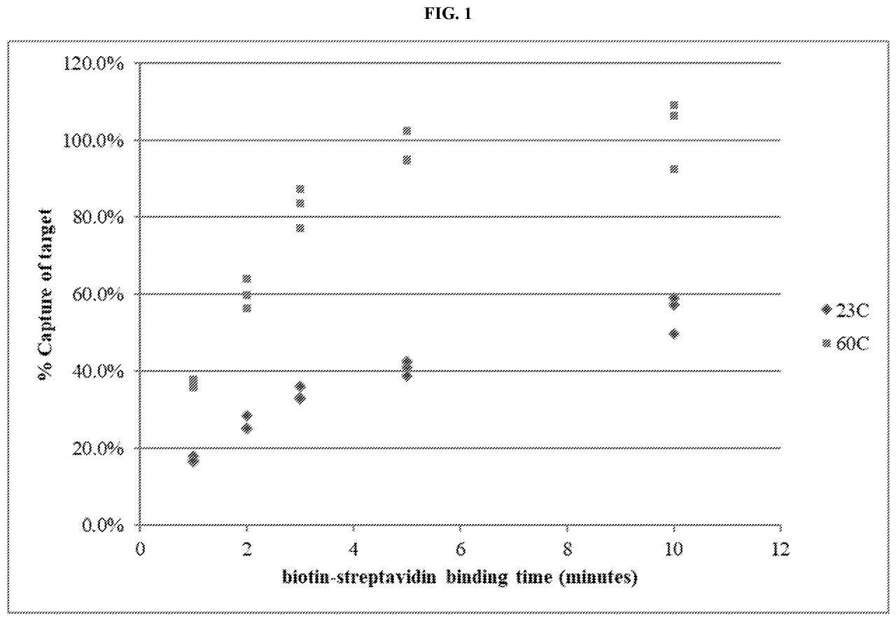

The biotin-streptavidin binding step was performed for 1, 2, 3, 5, or 10 minutes in the Benchmark heater-shaker set to 60° C., fitted with custom aluminum block that fits the 1.5 ml tubes snugly versus the same time intervals, but the tubes were incubated at room temperature (23° C.). All conditions were performed in triplicate. The biotin-streptavidin interaction step performs significantly better at 60° C. compared to room temperature in terms of overall yield as well as speed (FIG. 1).

Biotin-Streptavidin Interaction Vs. Temperature

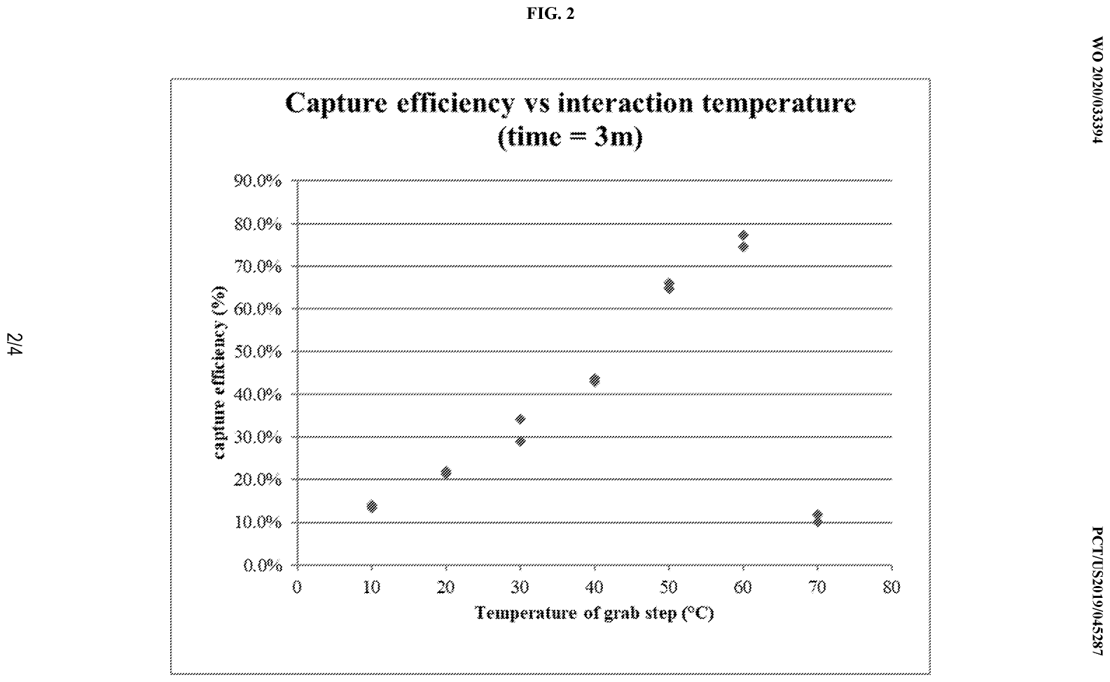

Biotinylated ORF3 capture probes 1 and 2 were hybridized to the specimen DNA at 60° C. for five minutes. The Biotin-streptavidin binding step was then performed for three minutes in the Benchmark heater-shaker set to 10° C., 20° C., 30° C., 40° C., 50° C., 60° C., or 70° C. A 10-minute binding step performed in the end-over-end rotator at room temperature served as a control/comparator to previous experiments. All conditions in duplicate. Performance of biotin-streptavidin binding step increases with temperature up to 60° C., and drops off above 60° C. (FIG. 2).

### Specific Capture/Biotin-Streptavidin Interaction at 60° C. has Decreased Nonspecific DNA Binding

To test if nonspecific DNA was co-extracted at different levels when biotin-streptavidin binding occurred at 20° C., 40° C. or 60° C., experiments were conducted during development of embodiments herein to measure the amount of human DNA on PMPs using human single-copy β-globulin gene, BGA assay (Abravaya, 2010; herein incorporated by reference in its entirety) while selectively capturing CT DNA. In this study, oligos were hybridized to target at 60° C. for 1 minute. When hybridization was complete, the tubes were placed in 20° C. heater-shaker to cool to room temperature before the PMPs were added, and the tubes were then placed at 20° C., 40° C., or 60° C. and incubated for 10 minutes. Extractions were evaluated with CT ORF3 and BGA qPCR reactions. As the temperature of the biotin-streptavidin binding step increases, the nonspecific DNA measured by a single copy human gene carried over decreases. Approximately 100 times (˜6 Cq) more nonspecific DNA is carried over with the PMPs when the streptavidin-biotin binding step is performed at 20° C. vs 60° C., and about twice as much CT DNA (1 Cq) was captured at 60° C. as at 20° C. (Table 2).

TABLE 2
Streptavidin-biotin binding temperature vs. Cq of CT and BGA.

| Binding | CT | | BGA | |
|---|---|---|---|---|
| temp ° C. | Cq | s.d. | Cq | s.d. |
| 20 | 20.84 | 0.05 | 29.95 | 0.12 |
| 40 | 19.92 | 0.12 | 32.34 | 0.42 |
| 60 | 19.9 | 0.07 | 35.84 | 1.25 |

### Streptavidin Binding at High Temperatures

Experiments conducted during development of embodiments herein demonstrate that streptavidin binding is not abolished at 70° C.; rather, the loss of apparent binding at 70° C. was a result of the Tm of the probe.

### Oligos

ORF3 cap probe 1 low Tm- 5′-biotin-AAA AAG GAT TAC
CTA TAA CTG TAG ACT CGG CTT GGG-3′
(SEQ ID NO: 1; Tm = 74.8° C. nearest neighbor with
300 mM NaCl)
ORF3 cap probe 2 low Tm- 5′-biotin-AAA AAC GCT CAA
ATC ATC GAG GAA AAC CGT ATG AG-3′
(SEQ ID NO: 2; Tm = 76.2 nearest neighbor with 300
mM NaCl)
ORF3 cap probe 2 high Tm - 5′-biotin-AAA AAA GCG
CTA CAC ACG CTC AAA TCA TCG AGG AAA ACC GTA TGA
GAA ACG GAT CTA AGC-3′
(SEQ ID NO: 3; Tm = 87.3 C. nearest neighbor with
300 mM NaCl)

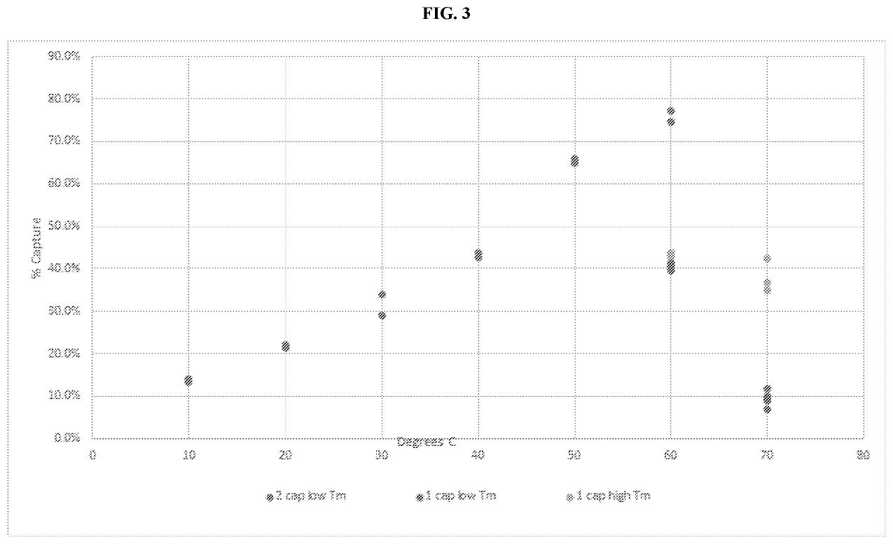

Results are depicted in FIG. 3. The “2 cap low Tm probe mix” had ORF3 cap probe 1 low Tm and ORF3 cap probe 1 high Tm. The “1 cap low Tm probe mix” had ORF3 cap probe 2 low Tm, and the “1 cap high Tm probe mix” had ORF3 cap probe 2 high Tm. The 1 cap probe mixes gave reduced capture due to the fact that they only contain one capture probe. However, the capture yield is the same at 60° C. and 70° C. when using the higher Tm capture probe (gray). Therefore, the loss of binding when at 70° C. of the low Tm probe mixes (blue and orange) is due to poor hybridization.

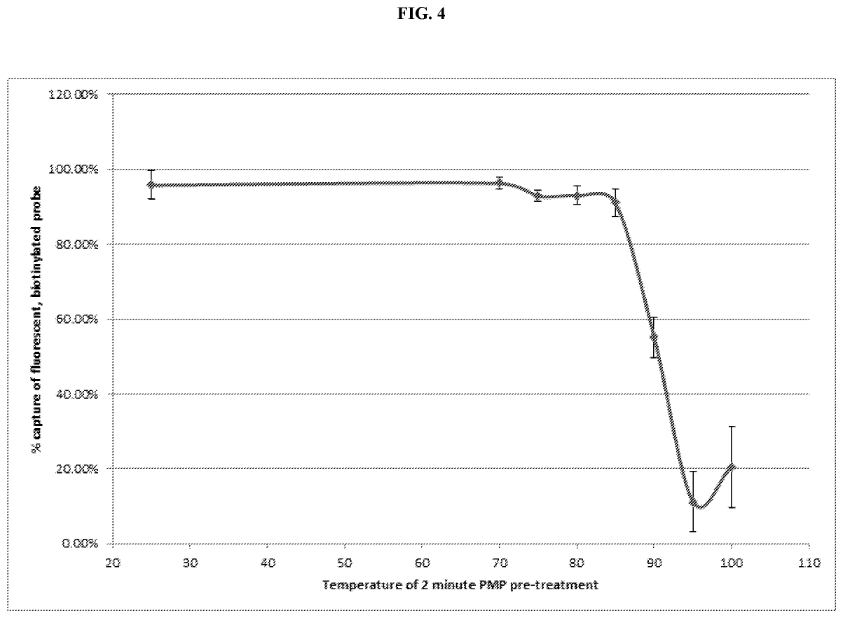

Experiments were conducted during development of embodiments herein to determine the temperature at which biotin-streptavidin bond fails to form. Streptavidin PMPs were pretreated to a range of temperatures and then measured the amount of a biotinylated FAM-labeled probe was bound (TT buffer: 10 Mm Tris pH 8.0 & 0.001% Tween 20; Binding Reaction Buffer: 1% SDS, 30 mM Tris pH=8, 300 mM NaCl, 80 mM MgCl₂). 100 μg streptavidin M270 PMPs were washed three times in U buffer. The PMPs were resuspended in 25 μl of U buffer and were either not heat treated (control) or heat treated at 70, 75, 80, 85, or 90° C. for 2 minutes. The PMPs were pelleted magnetically and the U supernatant was removed. To each 100 ug of washed and heat treated M270 Streptavidin PMPs, 5 ul of 333 nM biotin-cap-FAM probe (5′biotin-AAA AAC GAT CAA GGA GU CU CGG CAC CAG-FAM-3′ (SEQ ID NO: 4)), and 100 uL of binding reaction buffer were added. Reaction was incubated at 60° C. with constant shaking. The PMPs were pelleted magnetically and 25 μl of supernatant was pipetted into PCR tubes and read in FAM channel in Rotorgene Q. 0% binding was buffer plus probe without PMPs and 100% binding was buffer, probe and streptavidin M270 PMPs not pretreated. The experiments demonstrate that streptavidin loses its biotin binding activity when it is exposed to temperatures above 85° C. (FIG. 4).

### REFERENCES

The following references, which are cited above, are herein incorporated by reference in their entireties.
Green N M. Avidin and Streptavidin. Methods in Enzymology. 1990, 184:51-67.
Abravaya K, Erickson B J, Huang S X, Mak W B X, Salituro J A, Tang N. Primers and probes for detecting human papillomavirus and human beta globin sequences in test samples. U.S. Pub No. 2010/0081124.

1. A method of capturing a target nucleic acid, comprising:
(a) contacting a sample comprising the target nucleic acid and with an affinity-tagged capture oligonucleotide, wherein the capture oligonucleotide is complementary to all or a portion of the target nucleic acid;
(b) incubating the sample under conditions that allow a capture/target complex to form between the capture oligonucleotide and the target nucleic acid;
(c) contacting the sample and affinity-tagged capture oligonucleotide with a capture agent, wherein the capture agent and the affinity-tag are capable of forming a stable non-covalent complex;
(d) incubating the sample at a temperature of 30-85° C.; and
(e) separating the capture agent from the sample, thereby removing capture/target complex from the sample.

2. The method of claim 1, wherein the affinity tag comprises biotin.

3. The method of claim 2, wherein the capture agent comprises streptavidin.

4. The method of claim 1, wherein step (b) is performed at a temperature of less than 75° C.

5. The method of claim 1, wherein the capture agent is immobilized on a solid support.

6. The method of claim 5, wherein the capture agent is covalently linked to the solid support.

7. The method of claim 5, wherein the solid support is selected from the group consisting of a well, a tube, a plate, a chip, a bead, a particle, a membrane and a matrix.

8. The method of claim 7, wherein the solid support is a paramagnetic particle (PMP).

9. The method of claim 1, wherein the capture oligonucleotide is partially complementary to all or a portion of the target nucleic acid.

10. The method of claim 1, wherein the incubating of step (d) is carried out for at least 30 seconds.

11. The method of claim 10, wherein the incubating of step (d) is carried out for 30 seconds to 48 hours.

12. The method of claim 1, further comprising washing the capture/target complex following step (e).

13. The method of claim 1, further comprising eluting the target nucleic acid from the capture/target complex following step (e).

14. A method of capturing a target nucleic acid, comprising:
(a) contacting a sample comprising the target nucleic acid and with a biotin-tagged capture oligonucleotide, wherein the capture oligonucleotide is complementary to all or a portion of the target nucleic acid;
(b) incubating the sample under conditions that allow hybridization of the capture oligonucleotide to the target nucleic acid;
(c) contacting the sample and biotin-tagged capture oligonucleotide with streptavidin immobilized on a solid surface;
(d) incubating the sample at a temperature of 30-85° C. for sufficient time to allow formation of a biotin/streptavidin complex;
(e) separating the solid surface from the sample; and
(f) washing the solid surface to remove residual sample and/or contaminants.

15. The method of claim 14, wherein step (b) is performed at a temperature of less than 75° C.

16. The method of claim 14, wherein the streptavidin is covalently linked to the solid surface.

17. The method of claim 14, wherein the solid surface is selected from the group consisting of a well, a tube, a plate, a chip, a bead, a particle, a membrane and a matrix.

18. The method of claim 17, wherein the solid surface is a paramagnetic particle (PMP).

19. The method of claim 14, wherein the capture oligonucleotide is partially complementary to all or a portion of the target nucleic acid.

20. The method of claim 14, wherein the incubating of step (d) is carried out for at least 30 seconds.

21. The method of claim 20, wherein the incubating of step (d) is carried out for 30 seconds to 48 hours.

22. The method of claim 14, further comprising:
(g) eluting the target nucleic acid.

23. The method of claim 14, further comprising:
(h) amplifying the target nucleic acid.

24. The method of claim 14, further comprising:
(g) amplifying the target nucleic acid.
